(19) United States
(12) Patent Application Publication
(10) Pub. No.: US 2007/0239020 A1
(43) Pub. Date: Oct. 11, 2007

(54) ULTRASONOGRAPHY APPARATUS

(76) Inventors: Kazuhiro Iinuma, Nasushiobara-shi (JP); Yasutsugu Seo, Otawara-shi (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 11/624,442

(22) Filed: Jan. 18, 2007

(30) Foreign Application Priority Data
Jan. 19, 2006 (JP) 2006-011677

(51) Int. Cl. A61B 8/14

(52) U.S. Cl. 600/459

(57) ABSTRACT

An ultrasonic array probe is fixed to a rotational shaft at a predetermined angle, and thus a mechanical structure is made simple. An ultrasonic beam is electronically controlled so that an ultrasonic transmission/reception direction may become substantially perpendicular to the surface of the mamma. Thereby, data on the entire mamma including a C′ region can be collected only by the rotation of the probe. In addition, a membrane which is interposed between the probe and the mamma is formed to have a mesh-like structure, thereby reducing multiple reflection. Moreover, a B mode image and a C mode image are displayed at the same time, and thereby an accurate diagnosis can be performed in a short time.

### CROSS-REFERENCE TO RELATED APPLICATIONS

This application is based upon and claims the benefit of priority from prior Japanese Patent Application No. 2006-011677, filed Jan. 19, 2006, the entire contents of which are incorporated herein by reference.

### BACKGROUND OF THE INVENTION

1. Field of the Invention

The present invention relates generally to an ultrasonography apparatus for diagnosing mammary gland diseases, and more particularly to an ultrasonic mammary examination apparatus which is also usable for breast cancer screening.

2. Description of the Related Art

The disease rate of breast cancer in Japan is highest in people in their late forties. In 2004 the breast cancer screening by clinical breast examination alone was abolished, and breast cancer screening by X-ray mammography for people in their forties was begun (see Notification No. 0427001 (2004) issued by the Director of the Division of the Health for the Elderly, the Ministry of Health, Labour and Welfare). However, except for minute calcification in which X-ray attenuation is great, the contrast of an X-ray image of a soft tissue of a living body, which is acquired without using a contrast medium, is very weak, and it is pointed out that there is a high possibility of overlook. In mammography a subject experiences pain because the breasts are clamped between pressing plates during imaging. Thus, the mammography apparatus is not necessarily proper as a diagnosing apparatus.

On the other hand, an examination using ultrasonic, which is excellent in depicting a living soft tissue, has been practiced, and its effectiveness has been reported. This method, however, has not yet been popular in general. The main reason for this is that the examination by this method depends greatly on the ability and experience of paramedics. In the currently practiced ultrasonography, in usual cases, the paramedic manipulates an ultrasonic probe, moves the probe while putting it on the breast, and detects a cross-sectional region which appears to be abnormal. A cross-sectional image of the region that appears to be abnormal (this image is referred to as “tomogram”) is recorded, and a doctor will later view the tomogram for diagnosis. Since the probe is manually operated, the position of the cross section varies from time to time, and it is very difficult to obtain data having reproducibility. The probability of overlooking an abnormal region depends on the ability of the paramedic. In addition, the time that is needed for examining one subject is long, and it is difficult to examine many subjects in a short time.

On the other hand, various types of ultrasonography apparatuses, which are usable for diagnosis with least dependency on the paramedic's ability, have been proposed. Specifically, there have been proposed methods in which the probe is mechanically moved along a predetermined locus without manual operation by the paramedic, and ultrasonic data of the entire region of interest is collected and displayed as tomograms.

These methods are generally classified into a direct contact method and a water immersion method. In the direct contact method, the ultrasonic transmission/reception surface of the ultrasonic probe is put in contact with the surface of the body and tomograms in the body are displayed (see, e.g. Jpn. Pat. Appln. KOKAI Publication No. 2003-310614). In the water immersion method, a liquid, such as water, is interposed between the body surface and the wave transmission/reception surface of the ultrasonic probe, and in this state ultrasonic transmission/reception is executed. In the case of the direct contact method, there is no need to consider a problem of the effect of multiple reflection. However, the direct contact method has the following defect. That is, since the mamma is a soft tissue, if the probe contacts the mamma and moves, an image of the mammary tissue, which is obtained when the mammary tissue is deformed, becomes a deformed image that is different from an image obtained when the mammary tissue is in the static position. In the water immersion method, since the probe does not directly contact the mamma, there is little deformation of the mamma due to the movement of the probe. However, the water immersion method has such a defect that multiple reflection occurs between the wave transmission/reception surface of the probe and a membrane that is in contact with the mamma or the surface of the mamma, and an image due to multiple reflection mixes in a tomogram of the mammary tissue.

The water immersion method is further classified into a supine position method and a prone position method. In the supine position method, a subject lies on a subject table in the supine position and, in this state, a water bag is placed on the mamma and the probe in the water is mechanically moved (see, e.g. “Ultrasonic Diagnosis”, 2nd. Ed., the Japan Society of Ultrasonics in Medicine, 1994, p. 106). In the supine position method, the subject lies in the supine position, and this method is most natural for the subject. However, the probe and the probe driving mechanism, which are disposed in the water bag, are situated over the subject, and the entire structure needs to be moved. This results in complexity in structure, and there is no example of the use of an array probe in the prior art. A single transducer is mechanically reciprocated.

On the other hand, in the prone position method, a water bath and an ultrasonic probe are disposed in a hole formed in the patient table. The mamma is placed in the hole, and the probe is moved or rotated, thereby collecting data (see, e.g. Jpn. Pat. Appln. KOKOKU Publications No. S62-4989 and No. H4-14015). In this case, a complex structure is needed for varying the angle of the probe so as to make an ultrasonic beam incident on the body surface at right angles. Even if such a complex structure is adopted, there is such a disadvantage that if the ultrasonic beam is incident on the body surface at right angles, the effect of multiple reflection is considerably great and a high-quality image cannot be obtained. In addition, there is a disadvantage that a flat region that is called “C′” near the shoulder and axilla, where the ratio of occurrence of breast cancer is highest, cannot be depicted. Furthermore, in the conventional art, since the mamma is directly immersed in the water, the water becomes unclean and this method is not suited to examinations of many subjects.

Another serious problem in the ultrasonography apparatus is as follows. Since tomograms of the entire mamma, including not only a diseased part but also normal parts, are displayed, a great number of tomograms, for example, several-hundred tomograms, have to be acquired, and a heavy load is imposed on the doctor who diagnoses the mamma by viewing many tomograms. This point is a large difference from X-ray mammography in which both breasts can be diagnosed with four images in total. This point is a serious problem when the tomograms of the ultrasonography apparatus are used for the medical examination.

As has been described above, although various ultrasonography apparatuses, which are usable for breast cancer examination, have been proposed, there are many problems such as a time for examination, deformation of the breast, degradation in image quality due to multiple reflection, a complex driving mechanism, uncleanness of water, and a method of displaying many tomograms. In the prior art, none of such conventional ultrasonography apparatuses has been widely used as a practical apparatus.

### BRIEF SUMMARY OF THE INVENTION

The present invention aims at solving the problems in the prior art, and the object of the invention is to provide an ultrasonographic mammary examination apparatus which enables a good diagnosis of a lesion of the mamma without imposing a heavy load on subjects or paramedics.

According to an aspect of the present invention, there is provided an ultrasonography apparatus comprising: an ultrasonic probe which transmits an ultrasonic wave to a subject on the basis of a driving signal which is supplied, and generates an echo signal on the basis of a reflective wave from the subject, the ultrasonic probe being disposed in a liquid; an ultrasonic-transmissive membrane unit which is disposed between an ultrasonic wave transmission/reception surface of the ultrasonic probe and the subject and prevents contact between the liquid and the subject; a rotation mechanism which rotates the ultrasonic probe while the ultrasonic wave transmission/reception surface of the ultrasonic probe being opposed to the subject; a driving signal generating unit which generates the driving signal and supplies the driving signal to the ultrasonic probe; and a control unit which controls the rotation mechanism and the driving signal generating unit such that ultrasonic transmission/reception is executed while the ultrasonic probe is being rotated.

According to another aspect of the present invention, there is provided an ultrasonography apparatus comprising: an ultrasonic probe which transmits an ultrasonic wave to a subject, and generates an echo signal on the basis of a reflective wave from the subject, the ultrasonic probe being disposed in a liquid; a first membrane with ultrasonic transmissivity which is disposed between an ultrasonic wave transmission/reception surface of the ultrasonic probe and the subject and prevents contact between the liquid and the subject; and a second membrane which is formed integral with the first membrane and has a mesh-like structure for scattering the ultrasonic wave, thereby to prevent ultrasonic multiple reflection.

According to yet another aspect of the present invention, there is provided an ultrasonography apparatus comprising: an ultrasonic probe which transmits an ultrasonic beam to a subject by a plurality of ultrasonic transducers, and generates an echo signal on the basis of a reflective wave from the subject, the ultrasonic probe being disposed with a predetermined distance from the subject; and a control unit which controls a timing of supplying a driving signal to each of the ultrasonic transducers in accordance with a shape of the subject such that the ultrasonic beam is transmitted substantially perpendicular to a surface of the subject.

According to yet another aspect of the present invention, there is provided an ultrasonography apparatus comprising: an ultrasonic probe which transmits an ultrasonic wave to a subject, and generates an echo signal on the basis of a reflective wave from the subject, the ultrasonic probe being disposed with a predetermined distance from the subject; a rotation mechanism which rotates the ultrasonic probe while an ultrasonic wave transmission/reception surface of the ultrasonic probe being opposed to the subject; a control unit which executes ultrasonic transmission/reception while the ultrasonic probe is being rotated by the rotation mechanism, thereby acquiring ultrasonic data over at least 360° with respect to the subject; a data generating unit which generates voxel data in an orthogonal coordinate system by using the ultrasonic data over at least 360°; and an image generating unit which generates an ultrasonic image by using the voxel data.

### BRIEF DESCRIPTION OF THE SEVERAL VIEWS OF THE DRAWING

FIG. 1 is a block diagram showing the structure of an ultrasonography apparatus according to an embodiment of the present invention;

FIG. 2 shows an example of an ultrasonic scanning unit A;

FIG. 3A shows an ultrasonic scanning unit A according to Example 2;

FIG. 3B shows an ultrasonic scanning unit A according to Example 3;

FIGS. 4A and 4B show an example of an ultrasonic transmission membrane of a liquid sealing container;

FIGS. 5A and 5B are views for explaining the ultrasonic transmission membrane of the liquid sealing container;

FIGS. 6A and 6B are views for describing another example of the ultrasonic transmission membrane of the liquid sealing container;

FIG. 7 shows an example of the membrane structure;

FIG. 8 is a view for explaining multiple reflection;

FIG. 9 is a view for explaining reduction in multiple reflection;

FIG. 10 is a view for explaining an ultrasonic beam which is not perpendicular to a wave transmission/reception surface;

FIG. 11 is a view for explaining a multiplex reflection reduction effect of an ultrasonic beam which is emitted obliquely;

FIG. 12 is a view for explaining a case in which ultrasonic transmission/reception is executed in a plurality of directions;

FIG. 13 is a view for finding a formula which represents a position of a reflecting body by orthogonal coordinates;

FIG. 14 is a view which is taken in a z-axis direction in FIG. 13;

FIG. 15 is a view for explaining voxel data;

FIG. 16 is a view for explaining simultaneous display of a B mode and a C mode according to the embodiments shown in FIG. 2 to FIG. 3B; and

FIG. 17 shows an example in which an abnormal region is displayed by another cross section in the embodiments shown in FIG. 2 to FIG. 3B.

### DETAILED DESCRIPTION OF THE INVENTION

Embodiments of the present invention will now be described with reference to the accompanying drawings. In the description below, the structural elements having substantially the same functions and structures are denoted by like reference numerals, and an overlapping description is given only where necessary.

### First Embodiment

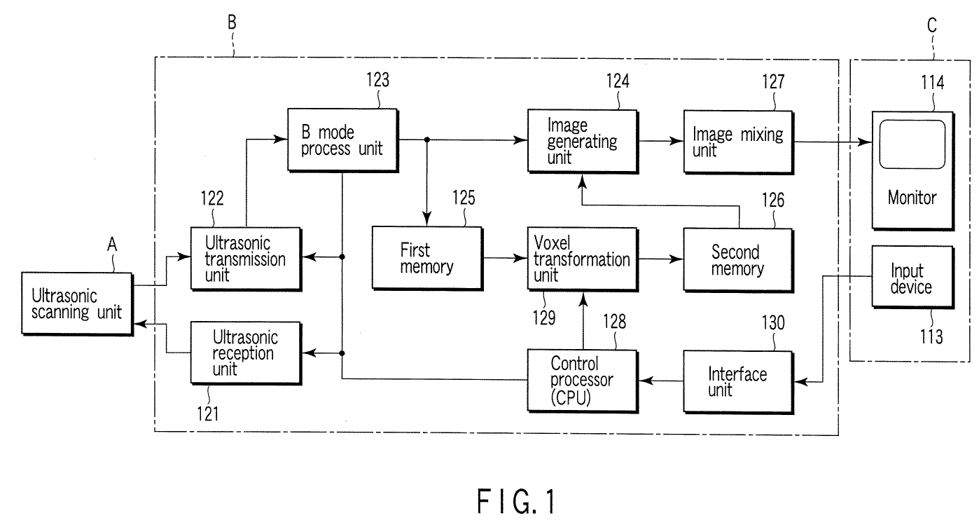

FIG. 1 is a block diagram showing the structure of an ultrasonography apparatus according to an embodiment of the invention. As shown in FIG. 1A, the ultrasonography apparatus includes an ultrasonography scanning unit A, an apparatus body B and a operation console C. The apparatus body B includes an ultrasonic transmission unit 121, an ultrasonic reception unit 122, a B mode process unit 123, an image generating unit 124, a first image memory 125, a second image memory 126, an image mixing unit 127, a control processor (CPU) 128, a voxel transformation unit 129, and an interface unit 130. In addition, the operation console C includes an input device 113 and a monitor 114.

The functions of the respective structural components will be described below.

The ultrasonic scanning unit A includes an ultrasonic array probe, a rotating mechanism which rotates the ultrasonic array probe while the ultrasonic transmission/reception surface of the ultrasonic array probe is being opposed to a subject, and a liquid container and the like. The specific structure of the ultrasonic scanning unit A will be described later in detail.

The input device 113 is connected to the apparatus body B and includes various switches, buttons, a track ball, a mouse and a keyboard for inputting to the apparatus body B from an operator, imaging conditions, scanning conditions, a display method, setting regions-of-interest (ROI), and instructions of setting various image quality conditions, and also includes a lever for instructing display modes such as a B mode image and a C mode image. The information input from the input device 113 is sent to the control processor 128 via the interface unit 113.

The monitor 114 displays, as an image, a combination of morphological information in a living body (e.g. B mode image, C mode image), a position information and a subject information on the basis of video signals from the image mixing unit 127.

The ultrasonic transmission unit 121 includes a trigger generating circuit, a delay circuit and a pulser circuit, which are not shown. The pulser circuit repeatedly generates rate pulses for forming transmission ultrasonic waves at a predetermined rate frequency fr Hz (cycle: 1/fr second). In addition, in the delay circuit, a delay time, which is necessary for converting ultrasonic waves in a beam shape on a channel-by-channel basis and determining a transmission directivity, is assigned to each rate pulse. At a timing based on the rate pulse, the trigger generating circuit applies a driving signal to each ultrasonic transducer of the probe 12. In addition, on the basis of a result of a calculation (to be described later) that is executed in order to make an ultrasonic beam incident substantially perpendicular to the surface of the mamma, the ultrasonic transmission unit 121 controls the timing of supplying a driving signal to each ultrasonic transducer.

The ultrasonic reception unit 122 includes an amplifier circuit, an A/D converter and an adder, which are not shown. The amplifier circuit amplifies an echo signal, which is taken in via the probe 12, on a channel-by-channel basis. The A/D converter imparts a delay time, which is necessary for determining reception directivity, to the amplified echo signal. Subsequently, the adder executes an addition process. By the addition process, a reflective component in a direction corresponding to the reception directivity of the echo signal is emphasized, and a comprehensive beam for ultrasonic transmission/reception is formed by the reception directivity and transmission directivity.

The B mode process unit 123 receives the echo signal from the reception unit 122, subjects the echo signal to logarithmic amplification and an envelope detection process, and generates data in which the signal intensity is represented by luminance. This data is stored in the first image memory 125 directly, and is sent to the image generating unit 124. In the image generating unit 124, a B mode image in which the intensity of the reflection wave is represented by luminance is generated. The B mode image is sent to the monitor 114 via the image mixing unit 127 and is displayed on the monitor 114.

The image generating unit 124 generated a B mode image, a C mode image, an arbitrary tomographic image and the like by using voxel data which is generated by the voxel transformation unit 126 according to an instruction via the input unit 113 and is stored in the second memory 126. In addition, the image generating unit 124 converts (scan-converts) a scanning line signal string of ultrasonic scan to a scanning line signal string of a general video format represented by, e.g. a TV video format, and generates an ultrasonographic image as a display image.

The voxel transformation unit 126 generates voxel data of an orthogonal coordinate system by using the ultrasonic data that is stored in the first memory 125 and is obtained by rotating the ultrasonic array probe in the liquid with executing ultrasonic scanning. The voxel data of the orthogonal coordinate system is stored in the second memory 126. The method of generating the voxel data will be explained later in detail.

The image mixing unit 127 mixes the image (or images) received from the image generating unit 124 with character information of various parameters, indices, etc., and outputs a video signal to the monitor 114.

The control processor 128 functions as an information processing unit (computer), and controls all operations with respect to the ultrasonic scanning unit A, the ultrasonography apparatus body B and the operation console C. In addition, the control processor 128 executes arithmetic operations, controls, etc. relating to various processes by using various purpose-specific programs (e.g. a phase calculation program for making an ultrasonic beam incident substantially perpendicular to the surface of the mamma, and a program for generating voxel data of the orthogonal coordinate system from the ultrasonic data obtained by a polar coordinate system), a control program for executing predetermined image generation/display, etc., from the internal storage unit 129.

The interface unit 130 is an device to send information input from the input device 113 to the control processor 128.

### [Ultrasonic Scanning Unit]

The structure of the ultrasonic scanning unit will be described in detail, with reference to Examples.

### EXAMPLE 1

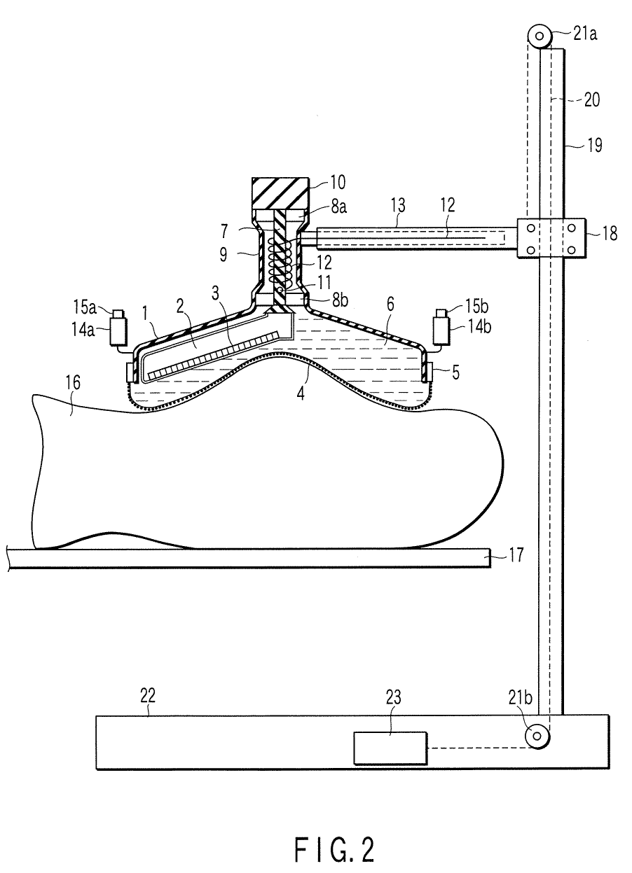

FIG. 2 shows an ultrasonic scanning unit A according to Example 1. A liquid sealing container, which contains hot water 6, is composed of a support cover 1, an ultrasonic transmission membrane 4 and a membrane fixing unit 5. An ultrasonic array probe 2 is disposed in the hot water 6. The liquid sealing container may be sealed completely or not. The ultrasonic array probe 2 is fixed to a rotational shaft 7 at a predetermined angle. The rotational shaft 7 is supported by bearings 8a and 8b. The rotational shaft 7 is rotated by a motor 10. Thereby, the ultrasonic array probe 2 is rotated in the liquid. The bearings 8a and 8b are fixed to an outer cylinder 9. The outer cylinder 9 is fixed to a distal end portion of a support arm 13 which is extendible. The other end portion of the support arm 13 is coupled to a support column 19 by means of a coupling unit 18. The support column 19 is secured to a support column base 22. Normally, hot water is used as the liquid. Although not shown, the hot water is circulated by a water supply/drain unit so that the temperature of water is kept at about 37° C. The temperature of the hot water in the container is measured by, e.g. a thermocouple and is always displayed.

The subject lies on a patient table 17 in the supine position, and the operator holds handles 14a and 14b which are fixed on the support cover 1 of the container. Making use of the push/pull operations of switches 15a and 15b, which are provided on upper parts of the handles 14a and 14b, and the handle 14b, the operator places the ultrasonic transmission membrane 4 of the liquid sealing container at a proper position on one of the breasts 16. Although the two handles 14a and 14b are situated substantially parallel to the body axis, FIG. 2 depicts the handles 14a and 14b as being perpendicular to the body axis for the purpose of description. If the switch 15a on the upper part of the handle 14a is pushed, an arm lock (not shown) is released and the support arm 13 is made extendible/contractible, and also a coupling unit lock (not shown) is released and the coupling unit 18 is made vertically movable and rotatable relative to the support column 19. In this state, the ultrasonic transmission membrane 4 of the liquid sealing container is positioned just above the breast. Further, if the handle 14b is turned to the far side while the switch 15b is being pushed, a wire 20 is pulled by a second motor 23 and the coupling unit 18 is raised via pulleys 21a and 21b. If the handle 14b is turned to the near side, the second motor rotates reversely to feed out the wire 20 and lower the coupling unit 18. Thus, by operating the handle 14b, the height of the container is properly set. If the switch 15a is released, the position of the container is fixed.

Array transducers 3 are arranged on the ultrasonic array probe 2 on the body surface side. A fine cable is connected to each of the transducers 3, and the fine cables of the transducers 3 are connected to a multicore cable 12 at the proximal end of the ultrasonic array probe 2. The multicore cable 12 extends through the inside of the rotational shaft 7 and is drawn out of the rotational shaft 7 via a hole 11 formed in the rotational shaft 7. A distal end portion of the multicore cable 12 passes through the arm 13 and is connected to the ultrasonic transmission unit 121 and the ultrasonic reception unit 122. The multicore cable 12 is wound several times around the rotational shaft 7 so as to be adaptive to the rotation of the rotational shaft 7. By the control of the ultrasonic transmission unit 121 and the ultrasonic reception unit 122, ultrasonic pulses which are emitted from the array transducers 3 propagate into the mammary tissue through the hot water 6 and ultrasonic transmission membrane 4 and are reflected in the mammary tissue. The reflective pulses are received by the array transducers 3 through the ultrasonic transmission membrane 4 and hot water 6. Each time the ultrasonic transmission/reception is executed, the transmission/reception direction of ultrasonic pulses (hereinafter referred to as “direction of ultrasonic beam”) is slightly shifted from the left to the right in FIG. 2 (this shift is referred to as “scan”) and image data of a cross section just under the array probe is collected. The collected data is recorded in the first memory and is displayed in real time for a test on the monitor 114. Further, the data recorded in the first memory is converted to voxel data (to be described later) in the voxel transformation unit 129 and recorded in the second memory. Using the voxel data, a tomogram which is suited to diagnosis is generated by the image generating unit 124, and the tomogram is displayed on the monitor 114.

In order to confirm that the liquid sealing container is placed at the proper position on the breast, a test scan button of the input device 113 on the operation console C is pressed, and the ultrasonic array probe is rotated once in about one second. Thereby, a rough image is produced and displayed. If the position of the liquid sealing container is not proper, the position is properly adjusted. Thereafter, the rotational scan button of the input device 113 is pressed, and tomographic data is collected over 360°. The collected data is recorded and displayed. In this way, 3D data is collected.

If the data collection relating to one breast 16 is completed, the liquid sealing container is placed on the other breast by the same method. Rotational scan is similarly executed, and data is collected, recorded and displayed. As regards both breasts, the time needed for setting the container is about 2 to 4 minutes and the time needed for rotational scan is about 20 seconds. The examination is completed within a net time of 5 minutes. During this time, the subject may simply lie on the patient table in the supine position, and the pain is not caused.

### EXAMPLE 2

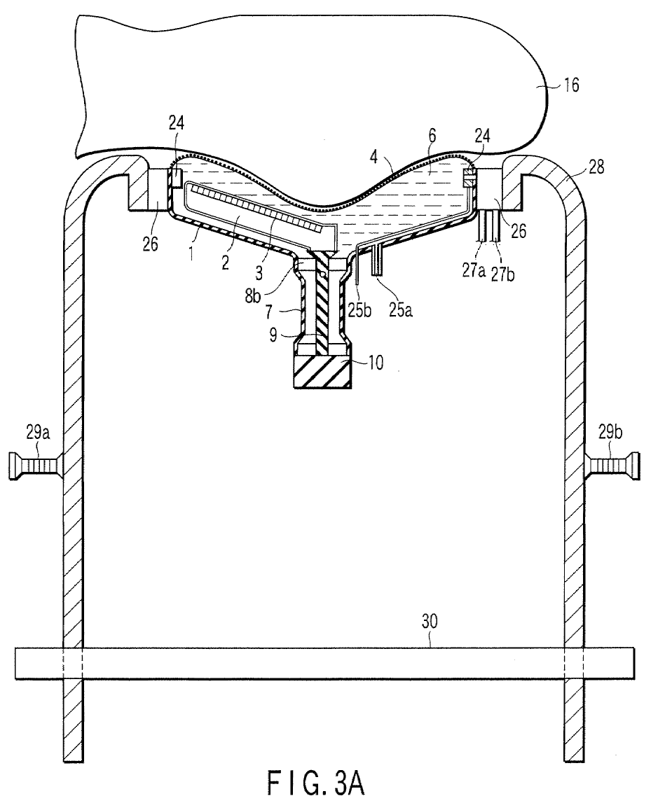

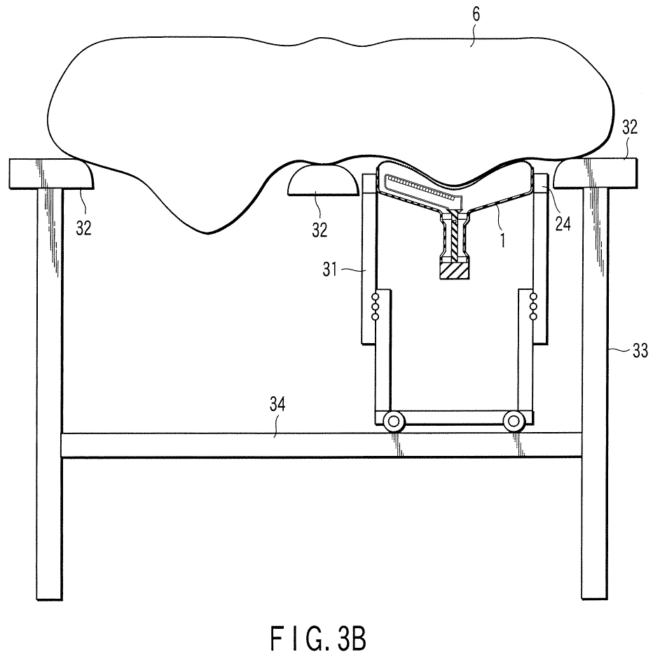

FIG. 3A shows an ultrasonic scanning unit A according to Example 2. Substantially the same liquid sealing container as that shown in FIG. 2 is disposed in an inverted fashion. The side surfaces of the support cover 1 are fixed to an outer frame 28 via a water supply/drain conduit 26. FIG. 2 depicts pipes 25a and 25b for circulating hot water at a fixed temperature of about 37° C. through the liquid sealing container, although the depiction of the pipes 25a and 25b is omitted in FIG. 2. Hot water at a fixed temperature circulates such that the hot water flows in through the intake pipe 25a, flows through the container, and flows out through the drain pipe 25b via a water port 24. The water port 24 is positioned above the liquid sealing container. Even if bubbles mix in the liquid sealing container, the bubbles are discharged from the water port 24 by the flow of the hot water.

The system according to Example 2 differs from the system according to Example 1 in that the water supply/drain conduit 26 is provided on the periphery of the support cover 1 of the liquid sealing container. Before the subject undergoes an examination, hot water at about 37° C. is supplied from a water supply pipe 27a to the water supply/drain conduit 26, and the conduit 26 is filled with the hot water. Further, the hot water flows over the ultrasonic transmission membrane 4 and covers the upper part of the ultrasonic transmission membrane 4. At this time, the drain pipe 27b is closed. In this state, if the subject puts the breast on the ultrasonic transmission membrane 4, the hot water on the membrane 4 fills a small gap between the membrane 4 and the surface of the breast, and the excess hot water overflows into the water supply/drain conduit and is drained through the drain pipe 27b. At this time, the drain pipe 27b is opened. The hot water, which is drained through the water supply/drain conduit 26, is isolated from the hot water 6 in the liquid sealing container. In this manner, by supplying and draining hot water over the ultrasonic transmission membrane 4, the presence of bubbles between the membrane 4 and the mammary surface is prevented. Since hot water that is in contact with the breast is replaced from subject to subject, and clean hot water is always kept. Besides, in Example 2, the subject, while standing, bends over and puts the breast on the ultrasonic transmission membrane 4 of the liquid sealing container. In order to stabilize the attitude, handles 29a and 29b, which are fixed to the outer frame 28, are provided and a footstool 30 is provided so as to be vertically movable in accordance with the body height.

### EXAMPLE 3

FIG. 3B shows an ultrasonic scanning unit A according to Example 3. In this ultrasonic scanning unit, a patient table is used for the outer frame 28 shown in FIG. 3A. The patient table has holes in regions corresponding to the breasts, and the subject lies prone on the patient table so that both breasts are put in the holes. FIG. 3B schematically shows a cross section 32 of the table with the holes, legs 33 for supporting the table, the same liquid sealing container as shown in FIG. 3A, a support frame 31 which supports the liquid sealing container, and a base 34 for moving the support frame 31. The liquid sealing container is moved just under one of the breasts with the movement of the support frame 31. Then, the liquid sealing container is vertically raised to push the breast, and stops there. The ultrasonic array probe rotates and collects image data. Thereafter, the liquid sealing container is once lowered, moved to a position just under the other breast, and raised once again to push the breast. Thus, the ultrasonic array probe collects image data, and the examination is completed.

### [Ultrasonic Transmission Membrane]

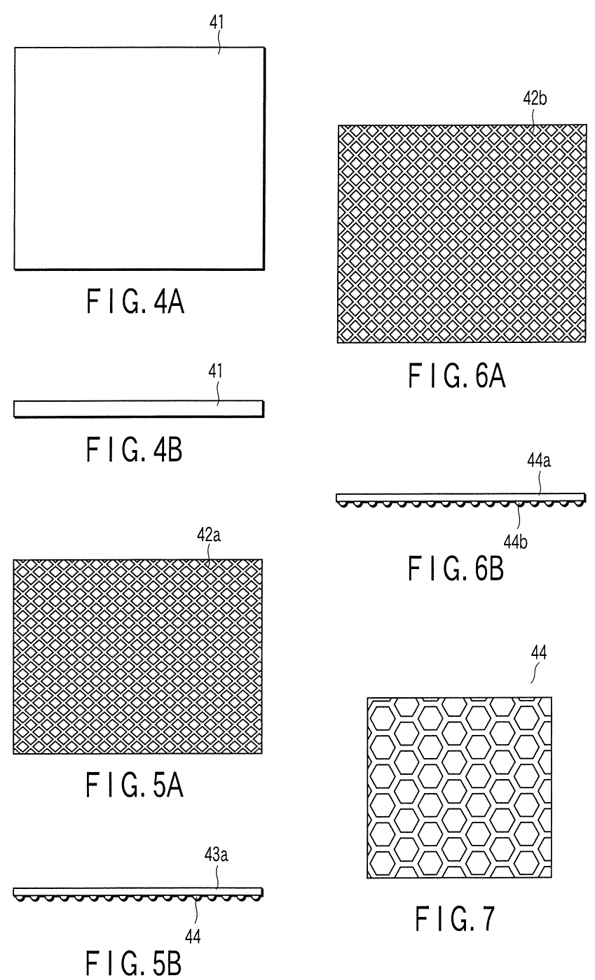

The structural components shown in the respective Examples will now be described in detail. FIG. 4, FIG. 5 and FIG. 6 show the structure of the ultrasonic transmission membrane 4 of the container shown in FIG. 2. An upper part of each Figure is a plan view of the membrane, and a lower part is a cross-sectional view of the membrane. FIG. 4 shows a conventional ultrasonic transmission membrane 41, which is formed of, e.g. a transparent vinyl sheet with a thickness of about 0.5 mm. The thickness of this membrane cannot greatly be reduced since the membrane transmits ultrasonic and needs to have such a strength as to bear the weight of the liquid in the container. Consequently, reflection and attenuation of ultrasonic due to the membrane are great.

FIG. 5 is a view for explaining a mesh-like membrane structure according to the embodiment. As shown in the cross-sectional view at the lower part of FIG. 5, an ultrasonic transmission membrane 42a having a mesh-like membrane structure comprises a waterproof, ultrasonic-transmissive thin sheet 43a and a mesh-like fabric 44a. Since the sheet 43a does not need to bear the weight of the liquid in the container, the sheet 43a is a very thin elastic soft membrane with a thickness of about 0.01 to 0.1 mm, which is formed of, e.g. vinyl or rubber. Under the sheet 43a, a mesh-like fabric, which has a high tensile strength and elasticity and is composed of fine fibers of about 0.5 mm, is provided to bear the weight of the liquid. FIG. 7 is an enlarged view of an example of the mesh-like structure. When the ultrasonic transmission membrane is placed on the breast, the mesh-like structure 44 deforms with its fibers varying not in length but in angle. Thus, the mesh-like structure 44 can easily extend and contrast in the vertical and horizontal directions, and can easily be put in close contact with the mammary surface. As shown in FIG. 1 or FIG. 2, the ultrasonic transmission membrane 4 is formed to have an average shape of the mamma. However, when the container is filled with liquid and placed on the mamma, the ultrasonic transmission membrane 4 extends/contracts to come in close contact with the mamma which varies in shape from subject to subject.

FIG. 6 shows another example of the ultrasonic transmission membrane. As shown in the cross-sectional view at the lower part of FIG. 6, the ultrasonic transmission membrane 42b having the mesh-like membrane structure includes a sheet 43b lying under a mesh-like fabric 44b. The mesh-like fabric 44b and sheet 43b are firmly attached to each other and formed as one piece. Another thin sheet may be provided on the mesh-like fabric 44b, and the sheets may be bonded or pressure-bonded in vacuum.

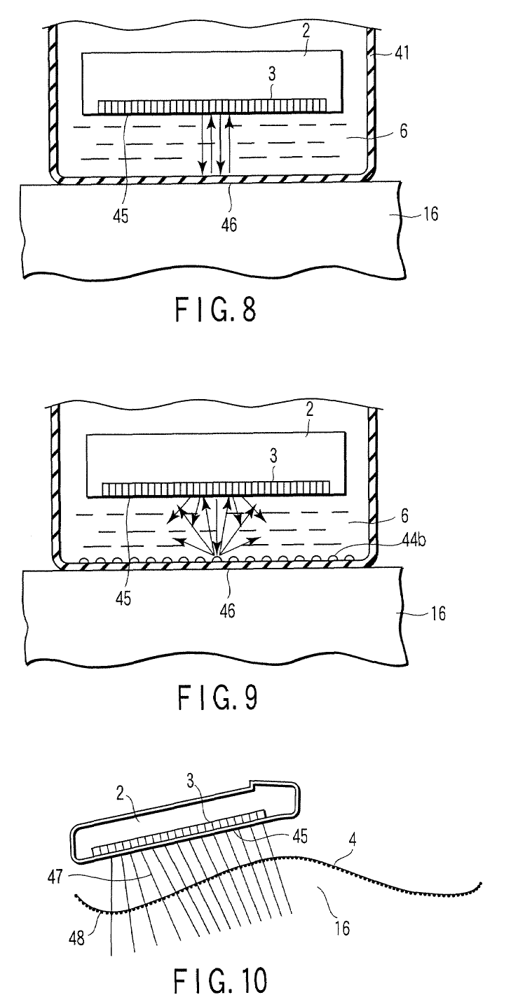

FIG. 8 and FIG. 9 are views for explaining how multiple reflection, which is the most serious problem in the water immersion method, is reduced by the mesh-like membrane structure. For easier description, FIG. 8 and FIG. 9 show the components in different shapes from those in FIG. 1, but the components denoted by common numerals represent substantially the same components. Multiple reflection, which adversely affects an image, occurs due to repetition of reflection of ultrasonic beams between a wave transmission/reception surface 45 of the ultrasonic array probe and the ultrasonic transmission membrane 4 of the container including the liquid or a surface 46 of the mamma 16. It is generally said that an ultrasonic beam should preferably be emitted perpendicular to the wave transmission/reception surface 45 of the probe and made incident perpendicular to the mammary surface 46, thereby to void refraction. FIG. 8 shows prior art. In FIG. 8, ultrasonic beams indicated by arrows propagate substantially perpendicular to the wave transmission/reception surface 45 and the ultrasonic transmission membrane 41 or mammary surface 46, and also the thickness of the ultrasonic transmission film 41 is great. Thus, the repetition of reflection is conspicuous.

FIG. 9 shows the case of the ultrasonic transmission film 42b having the mesh-like membrane structure shown in FIG. 6. The ultrasonic beams are scattered in various directions by the mesh-like structure 44b of the surface of the ultrasonic transmission film 42b. Since the degree of vertical reflection from the thin sheet is small, the multiple reflection can remarkably be reduced.

In applying the ultrasonic transmission membrane having this mesh-like membrane structure, there are no restrictions to the object of imaging or the type of ultrasonic probe which is used for imaging. The above-described advantageous effect can be obtained if this ultrasonic transmission membrane is applied to the imaging using the water immersion method.

### [Scanning Direction of Ultrasonic Beams]

FIG. 10 shows the directions of scanning of ultrasonic beams by the ultrasonic array probe 2. Normally, the array transducers 3 of the ultrasonic array probe 2 are arranged linearly, and the cross section of the wave transmission/reception surface 45 is linear. However, the mammary surface is a curved surface, and the cross section of the mammary surface is represented by a curve. The shape of the curve is not always an arc. In particular, a region 48 that is called “C′” near the axilla, where the ratio of occurrence of breast cancer is high, is substantially flat, and it is important to scan this region. If all the directions of ultrasonic beams are perpendicular to the wave transmission/reception surface and are parallel with each other, it is difficult to set the angles of incidence on the mammary surface 46 including the C′ region 48 at nearly right angles. In the present embodiment, the directions of beams are controlled by imparting phase differences to the oscillation elements of the ultrasonic array probe, and scanning lines 47 are not necessarily set in parallel and are set to be as much as possible perpendicular to the mammary surface. The scanning lines 47 in FIG. 10 are roughly depicted for the purpose of description. Actually, about 200 scanning lines are provided within a range of, e.g. 10 cm.

The phase difference, which is imparted to each oscillation element, can be calculated, for example, in the following manner. In the case where the mamma is scanned by ultrasonic beams while the ultrasonic array probe 2 is being rotated, the shape of the mamma is obtained at the scan operations to acquire the first image, for example, on the basis of the time and sound velocity between a time point at which the ultrasonic beam is transmitted in each scanning line and a time point at which a reflective wave with a predetermined intensity or more is first obtained. Based on the obtained shape of the mamma, the control processor 128 calculates the phase difference to be imparted to the ultrasonic pulse from each ultrasonic transducer (i.e. a delay time to be imported to the driving signal of each ultrasonic transducer) so that the ultrasonic beam, which is used in the second and following ultrasonic scan operations to acquire the second image subsequently, may be transmitted substantially perpendicular to the mammary surface. To obtain the third image, a delay time calculated on the basis of the second image. Since to rotating speed of the probe is low, an image is acquired each time the probe rotates approximately 1 degree. This way for image acquisition is sufficient since there is not a considerable difference between the adjacent images (such as the first and second images, or the second and third images). Thereby, in the ultrasonic scan operations at each rotational angle, the ultrasonic beam is transmitted substantially perpendicular to the mammary surface.

In the case where the direction of the ultrasonic beam is controlled so that the beam may be incident substantially perpendicular to the mammary surface 46, a multiple reflection reduction effect, which is different from the effect by the mesh-like structure membrane, is obtained. FIG. 11 is a view for explaining the effect of reducing multiple reflection. For the purpose of easier understanding, the wave transmission/reception surface 45 of the probe is depicted as being horizontal. When the ultrasonic beam is made perpendicularly incident on a part of the mammary surface 46, which is inclined to the wave transmission/reception surface 45, the ultrasonic beam, which has been perpendicularly incident on the mammary surface 46, is perpendicularly reflected, and reaches that part of the wave transmission/reception surface 45 of the probe, from which the beam is emitted. The ultrasonic beam, which has reached the wave transmission/reception surface 45, is reflected by the wave transmission/reception surface 45, not in a direction perpendicular to the wave transmission/reception surface 45 but in a direction of the angle of refection. This ultrasonic beam is reflected once again by the mammary surface 46 and reaches a part of the wave transmission/reception surface 45, which is away from the part from which the ultrasonic beam is emitted. Since the detection sensitivity of the part, where the ultrasonic beam reaches, is very low, the multiple reflection can greatly be reduced.

One scanning line is obtained by executing transmission/reception of the ultrasonic beam in one direction. By slightly shifting the scanning line within the cross section, one image (one frame) is formed of a plurality of scanning lines. The time that is needed for obtaining one scanning line is substantially equal to the time during which the ultrasonic pulse reciprocates over the distance corresponding to the depth of the viewing field in the cross section within the body. For example, in the case where the depth of the viewing field is 10 cm (the reciprocal distance is 20 cm), the scanning line interval is 1 mm and one image is formed of 100 scanning lines, the time needed for forming one image is 100×(2×0.1 m)/(1500 m/s)≅0.013 s since the sound velocity of ultrasonic (propagation speed) is about 1500 m/s. If the ultrasonic array probe is rotated and one image is obtained each time the ultrasonic array probe rotates by one degree, the time needed for a single rotation (360°) is 0.013 s×360=4.8 s, and the scanning of one of the breasts is completed in 4.8 seconds. In other words, all 3D data of one of the breasts is obtained in about 5 seconds. If a 2-directional simultaneous reception scheme, in which two scanning lines are generated by processing reception signals, is adopted instead of the 1-directional transmission/reception, one image can be generated by 200 scanning lines with a double scanning line density in the same time period.

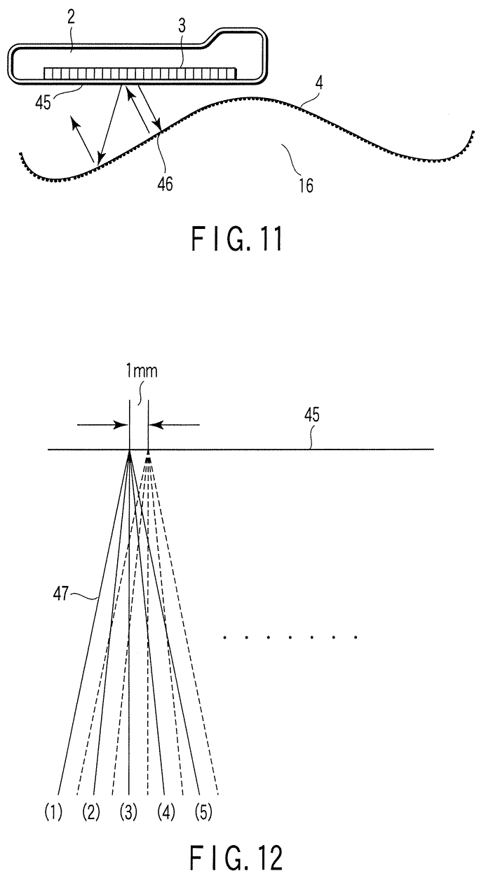

FIG. 12 illustrates a method of collecting still more information. Instead of executing transmission/reception of the ultrasonic beam in one direction, the transmission/reception of the ultrasonic beam is executed, for example, in five directions with different angles and then the beam is shifted by 1 mm. Repeatedly, the transmission/reception of the ultrasonic beam is executed in five directions and then the beam is shifted by 1 mm. Although the scanning time increases 5 times and the scanning of one of the breasts in the above-described example requires 4.8×5=24 s, i.e. 24 seconds, information of reflective waves in different transmission/reception directions can be obtained.

### [Collection of Image Data, Image Generation and Display]

Next, methods of collecting image data and generating and displaying an image are described in detail.

The first method is a simplest method. A reflective signal intensity of a received ultrasonic wave is stored in the first memory 125, and a tomographic image is displayed on the monitor 114 in real time. As the probe rotates, the tomographic image varies. If the probe rotates over 360°, data of all cross sections can be collected and displayed. This method is excellent in terms of simplicity. In particular, this method is used for confirming whether data collection is properly performed.

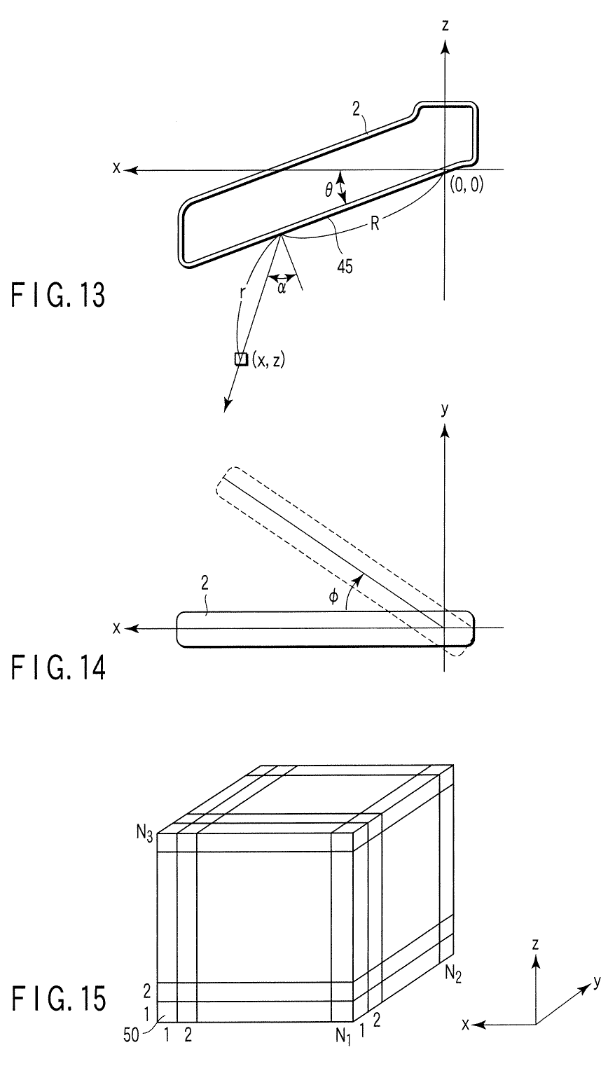

The second method is a display method which the doctor uses for diagnosis. All data of one of the breasts, which is obtained by a single rotation of the probe, is recorded in the first memory 125. This data is converted to voxel data of a three-dimensional (3D) orthogonal coordinate system by the voxel transformation unit 129, and the converted data is recorded. FIG. 13 shows the ultrasonic probe, which is shown in FIG. 2, FIG. 3A or FIG. 3B, and its rotational axis. The rotational axis is set as a z axis. A point at which the wave transmission/reception surface of the probe intersects the z axis is set as the origin. A direction, which extends through the origin and intersects the z axis at right angles, is an x axis. FIG. 13 shows the case in which the probe is set in a direction perpendicular to the start position of rotation, that is, the body axis. Assume now that at a point of a distance R from the origin along the wave transmission/reception surface of the probe, ultrasonic transmission/reception is executed at an angle α in a direction perpendicular to the wave transmission/reception surface, and a reflective signal is received from a point (x, z) at a distance of a depth r. The coordinates (x, z) of this point are expressed by

x=(R+r sin α) cos θ−r cos α sin θ (formula 1)

z=(R+r sin α)sin θ+r cos α cos θ (formula 2)

In the formulae, θ is a value that is predetermined in the apparatus, R and α are values which are known from control signals for transmission/reception, and r is calculated from the propagation time in which the ultrasonic wave reciprocates over the distance of depth r. FIG. 14 is a top view which is taken from above in FIG. 13, and illustrates the probe which rotates about the z axis. If an axis, which extends through the origin and intersects the x axis at right angles, is an y axis, the position coordinates (x, y, z) of the reflective wave from the depth r at the distance R and angle α, in the case where the probe is rotated by φ from the start position, are expressed by

x=[(R+r sin α)cos θ−r cos α sin θ]cos φ (formula 3)

y=[(R+r sin α)cos θ−r cos α sin θ]sin φ (formula 4)

z=(R+r sin α)sin θ+r cos α cos θ. (formula 5)

The reflective signal intensity of the ultrasonic wave reflected from the point at coordinates (x, y, z), together with the value of (R, r, α, φ), is first recorded in the first memory 125. Then, the reflective signal recorded in the first memory 125 is converted to orthogonal coordinates (x, y, z) according to formulae 3, 4 and 5 for coordinate conversion by the voxel transformation unit 129, by using the value of (R, r, α, φ). The converted orthogonal coordinates are recorded in the second memory 126. The coordinates recorded in the second memory 126 are voxel data shown in FIG. 15. The number of voxels, as shown in FIG. 15, is a finite value, i.e. N1, N2 and N3 in x, y and z directions, and is not a continuous value. If two or more data are contained in one voxel, a mean value of the data is used. As regards a voxel containing no data, a mean value of data of neighboring voxels is substituted. In this manner, all 3D data are converted to orthogonal coordinates and recorded in the memory. Thereby, the 3D voxel data are obtained as shown in FIG. 15. Using the obtained voxel data, it is possible to implement an arbitrary display method, such as a B mode, a C mode, a composite image, and a 3D image. In the above-described example, if scanning is executed for one point in five directions, five sets of voxel data are collected.

In a concrete display method, as a first method, a vertical cross-sectional display called “B mode”, that is, a cross section parallel to the z axis, is executed. A great number of cross sections constituting the entire mamma are successively displayed while the probe is being moved in parallel to the body axis or is being rotated about the z axis. A display mode selection button is provided on the input device 113 of the operation console C. The display method is selected by operating the selection button, and tomographic images are successively displayed by operating a lever of the input device 113. If the lever is turned from the center position to the far side, cross sections are successively displayed. If the lever is turned from the center position to the near side, the cross sections are displayed in the reverse order. By the angle of the lever, the display speed can be varied stepwise or continuously. ID information, which can identify the position of the cross section, is recorded on each tomographic image. The ID information and the position of the tomographic image, which is automatically obtained from the ID information, are superimposed on the current tomographic image in the image mixing unit 127 are displayed on the same screen. For example, when the position of a tomographic image to be displayed is moved in the body axis direction, the “left” or “right” of the breast is expressed by “L” or “R”, and the z-axis coordinate is expressed by a numerical value. When the tomographic image is to be displayed by moving the position thereof in the rotational direction, the rotational angle φ is recorded and displayed, and the position of the cross section is displayed by a straight line on the pattern of the breast which is simulated by a circle. When an abnormal region is found, the lever is adjusted to display an optimal cross section and the cross section is recorded as a still image. Since ID information is also recorded on this image, the ID information may be designated, for example, at the time of re-examination. Thereby, the image of this region can easily be reproduced from the recorded 3D data.

In another method, a cross section perpendicular to the z axis, which is normally called “C mode”, is displayed. A plurality of cross sections are successively displayed while the cross section is being moved in the z direction. The operation method for observing the image is the same as that in the case of B mode. In the C mode, since the number of images is relatively small, the images may be displayed on one screen in an arranged fashion or may be displayed on a photographic film.

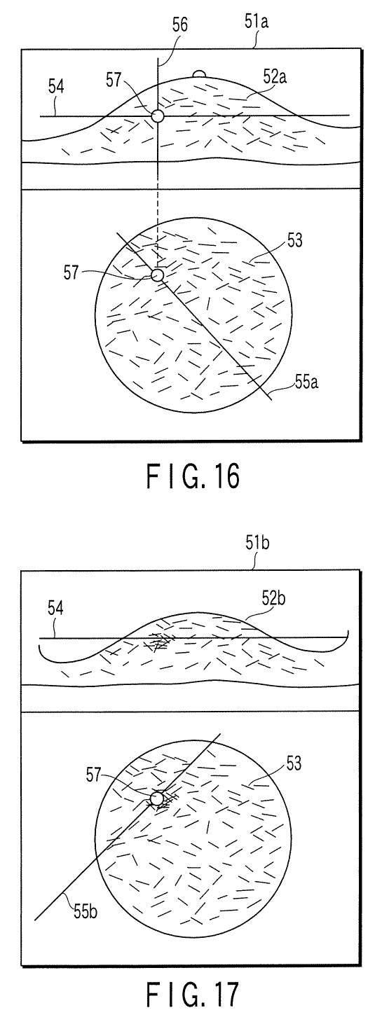

The method, which is considered most desirable, is a method in which both B-mode image and C-mode image are displayed on one screen in an arranged fashion, and one of these images is moved and the position thereof is displayed on the other image by a marker. FIG. 16 shows an example of this display method. A B mode image 52a is displayed on the upper part of the monitor 114, and a C mode image 53 is displayed on the lower part of the monitor 114. The C mode image is a horizontal tomographic image which is parallel to the body axis. The position of the displayed cross section is displayed as a horizontal line 54 in the B mode image that is displayed on the upper part. On the other hand, it is understood that the B mode image, which is displayed on the upper part, is a cross section that is displayed by a straight line 55a on the C mode image displayed on the lower part. If an abnormal region 57 is found, the optimal cross section for observation is displayed in a frozen state, and a vertical straight line 56 is aligned with the abnormal region 57 on the B mode image and then the lever is operated. Thereby, as shown in FIG. 17, B mode images, which are rotated about the straight line 56, are successively displayed on the upper part of the screen, and the position of the displayed cross section is displayed as a straight line 55b in the C mode image that is displayed on the lower part. By this display method, the doctor can diagnose many tomographic images in a short time in detail.

In still another display method, in the example shown in FIG. 12 in which the scanning direction for one point is changed to, e.g. five directions, the five kinds of image data in the respective directions are added and displayed. If a particularly detailed observation is to be desired, the image is frozen at this time, and the images of the cross section in the plural directions are displayed on the same screen and compared. In the added image, speckles and multiple reflection are reduced, and a signal-to-noise ratio is improved, and an smooth image, which is easy to view, is provided. Images, which are acquired by transmitting/receiving the beam in different directions, have different information, and these images are useful for more accurate diagnosis.

According to the above-described embodiments, the structure in which the ultrasonic array probe is fixed to the rotational shaft in the liquid is adopted. Thus, the structure of the embodiment is very simple. Simply by rotating the ultrasonic array probe by electronically controlling the transmission/reception direction of the ultrasonic beam, the ultrasonic beam can be transmitted/received in the as much as possible perpendicular direction to the mammary surface, and the multiple reflection can be reduced. Furthermore, the scanning of the region C′, which is difficult to perform, is enabled, and 3D data of the fixed mamma can be collected in a short time and various display methods that are suited to diagnosis can be used.

Since the structure in which the liquid sealing container is moved and set at a proper position of the mamma is adopted, the subject can be examined in the supine position that is the most desirable attitude.

A liquid, which is separate from the liquid in the liquid sealing container, is supplied/drained to/from the upper part of the liquid sealing container, which is situated under the body surface. Thereby, no bubbles are present on the mammary surface, and a good image can be obtained. Moreover, the liquid in contact with the mamma hardly becomes unclean and is kept clean.

By the electrical control, the ultrasonic beam is transmitted/received in the as much as possible perpendicular direction to the mammary surface. Thereby, the structure is simplified, the multiple reflection can be reduced, and the scanning of the region C′, which is difficult to perform, is enabled.

The mesh-like membrane structure is adopted for the ultrasonic-transmissive membrane of the liquid sealing container. Thereby, the multiple reflection, which is the most serious problem in the water immersion method, can be reduced, the transmittance of ultrasonic can be enhanced, and the strength of the container can be secured.

If the image data obtained by executing ultrasonic transmission/reception in a plurality of directions are used, the multiple reflection can be reduced, the speckles can be reduced, the signal-to-noise ratio can be increased, and a high-quality image can be obtained. In addition, the amount of useful information for diagnosis is increased, and the depiction of factiferous ducts immediately under the nipple, which are normally considered difficult to view, is enabled.

By generating or mixing images which are obtained by scanning the same cross section in different directions, it becomes possible to display images having different diagnostic information between the transmission/reception directions. The multiple reflection can be confirmed and reduced, the speckles can be reduced, and the signal-to-noise ratio can be improved.

The image data that is collected over the entire mamma is converted to voxel data, and various images are generated from the voxel data. Thereby, with use of general-purpose hardware and software for image processing, images suited to diagnosis can easily be generated and displayed.

Furthermore, the B mode image and C mode image are displayed at the same time, and one of these images is successively switched and the position of the associated cross section is displayed. Thereby, several hundred tomographic images can be observed in a short time, and an abnormal region can be examined in detail.

The cross-sectional position information is recorded on each tomographic image, and the cross-sectional position is automatically displayed by the marker on the screen on the basis of the recorded information. Thereby, the position of the displayed cross section can intuitively be recognized, the search of the cross section to be displayed is facilitated, and the necessary image can easily be displayed on the screen.

The present invention is not limited directly to the above-described embodiments. In practice, the structural elements can be modified without departing from the spirit of the invention. Various inventions can be made by properly combining the structural elements disclosed in the embodiments. For example, some structural elements may be omitted from all the structural elements disclosed in the embodiments. Furthermore, structural elements in different embodiments may properly be combined.

What is claimed is:

1. An ultrasonography apparatus comprising:
an ultrasonic probe which transmits an ultrasonic wave to a subject on the basis of a driving signal which is supplied, and generates an echo signal on the basis of a reflective wave from the subject, the ultrasonic probe being disposed in a liquid;
an ultrasonic-transmissive membrane unit which is disposed between an ultrasonic wave transmission/reception surface of the ultrasonic probe and the subject and prevents contact between the liquid and the subject;
a rotation mechanism which rotates the ultrasonic probe while the ultrasonic wave transmission/reception surface of the ultrasonic probe being opposed to the subject;
a driving signal generating unit which generates the driving signal and supplies the driving signal to the ultrasonic probe; and
a control unit which controls the rotation mechanism and the driving signal generating unit such that ultrasonic transmission/reception is executed while the ultrasonic probe is being rotated.

2. The ultrasonography apparatus according to claim 1, wherein the membrane unit has a shape for contact with a mamma of the subject.

3. The ultrasonography apparatus according to claim 1, wherein at least a part of the membrane unit has elasticity.

4. The ultrasonography apparatus according to claim 1, wherein the membrane unit comprises:
a first membrane with ultrasonic transmissivity and a water-proof property; and
a second membrane having a mesh-like structure for scattering the ultrasonic wave, thereby to prevent ultrasonic multiple reflection.

5. The ultrasonography apparatus according to claim 1, further comprising a water supply/drain unit for supplying and draining the liquid.

6. The ultrasonography apparatus according to claim 1, further comprising a container for containing the ultrasonic probe and the liquid, and for placing a contact surface, which is formed by the membrane unit, on an upper side of the subject.

7. The ultrasonography apparatus according to claim 1, further comprising a container for containing the ultrasonic probe and the liquid, and for placing a contact surface, which is formed by the membrane unit, on a lower side of the subject.

8. The ultrasonography apparatus according to claim 1, wherein an angle between the ultrasonic transmission/reception surface of the ultrasonic probe and a rotational axis of the rotation is not a right angle.

9. The ultrasonography apparatus according to claim 1, further comprising a calculation unit for calculating a delay time of each of the driving signals for ultrasonic transducers of the ultrasonic probe in accordance with a shape of a surface of the subject,
wherein the control unit controls the driving signal generating unit such that each driving signal is supplied to each ultrasonic transducer in accordance with the calculated delay time.

10. The ultrasonography apparatus according to claim 9, wherein the calculation unit calculates the delay time of the driving signal for each ultrasonic transducer of the ultrasonic probe such that the ultrasonic wave is transmitted in a substantially perpendicular direction to the surface of the subject.

11. The ultrasonography apparatus according to claim 1, further comprising a unit which generates or mixes images which are obtained by scanning the same cross section in different directions.

12. The ultrasonography apparatus according to claim 1, further comprising a unit which converts collected image data to voxel data, generates various images from the voxel data, and selects and displays the images.

13. The ultrasonography apparatus according to claim 12, further comprising a display unit which displays a B mode image and a C mode image at the same time, displays one of the B mode image and C mode image by successively switching a cross section of said one image to different cross sections, and displays a position of one of the B mode image and C mode image on the other of the B mode image and C mode image.

14. The ultrasonography apparatus according to claim 12, further comprising a display unit which records cross-sectional position information on each of tomographic images, and automatically displays a cross-sectional position by a marker on a screen on the basis of the recorded cross-sectional position information.

15. The ultrasonography apparatus according to claim 13, further comprising a display control unit which has a function of a speed of successively switching the cross section to different cross sections stepwise or continuously, switching the cross sections in a forward direction or a reverse direction, and controlling freezing of the image.

16. The ultrasonography apparatus according to claim 1, further comprising a temperature display unit which displays a temperature of the liquid.

17. An ultrasonography apparatus comprising:
an ultrasonic probe which transmits an ultrasonic wave to a subject, and generates an echo signal on the basis of a reflective wave from the subject, the ultrasonic probe being disposed in a liquid;
a first membrane with ultrasonic transmissivity which is disposed between an ultrasonic wave transmission/reception surface of the ultrasonic probe and the subject and prevents contact between the liquid and the subject; and
a second membrane which is formed integral with the first membrane and has a mesh-like structure for scattering the ultrasonic wave, thereby to prevent ultrasonic multiple reflection.

18. An ultrasonography apparatus comprising:
an ultrasonic probe which transmits an ultrasonic beam to a subject by a plurality of ultrasonic transducers, and generates an echo signal on the basis of a reflective wave from the subject, the ultrasonic probe being disposed with a predetermined distance from the subject; and
a control unit which controls a timing of supplying a driving signal to each of the ultrasonic transducers in accordance with a shape of the subject such that the ultrasonic beam is transmitted substantially perpendicular to a surface of the subject.

19. An ultrasonography apparatus comprising:
an ultrasonic probe which transmits an ultrasonic wave to a subject, and generates an echo signal on the basis of a reflective wave from the subject, the ultrasonic probe being disposed with a predetermined distance from the subject;
a rotation mechanism which rotates the ultrasonic probe while an ultrasonic wave transmission/reception surface of the ultrasonic probe being opposed to the subject;
a control unit which executes ultrasonic transmission/reception while the ultrasonic probe is being rotated by the rotation mechanism, thereby acquiring ultrasonic data over at least 360° with respect to the subject;
a data generating unit which generates voxel data in an orthogonal coordinate system by using the ultrasonic data over at least 360°; and
an image generating unit which generates an ultrasonic image by using the voxel data.
